(19) United States
(12) Patent Application Publication
(10) Pub. No.: US 2004/0266796 A1
(43) Pub. Date: Dec. 30, 2004

(54) Convergent processes for the synthesis of a GARFT inhibitor containing a methyl substituted thiophene core and intermediates therefor

(75) Inventors: Elena Dovalsantos, San Diego, CA (US); Erik Jon Flahive, San Diego, CA (US); Brian John Halden, San Diego, CA (US); Mark Bryan Mitchell, San Diego, CA (US); Wolfgang Reinhard Ludwig Notz, La Jolla, CA (US); Stacy Ann O'Neill-Slawecki, San Diego, CA (US); Qingping Tian, San Diego, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/875,836

(22) Filed: Jun. 23, 2004

Related U.S. Application Data
(60) Provisional application No. 60/482,320, filed on Jun. 25, 2003.

(51) Int. Cl. A61K 31/519; C07D 487/02

(52) U.S. Cl. 514/264.11; 544/280

(57) ABSTRACT

The invention relates to processes for the preparation of a GARFT inhibitor containing a methyl substituted thiophene core having the following structure:

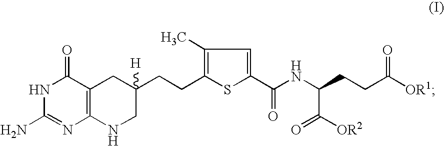

wherein each of R¹ and R² are independently a hydrogen atom or a moiety that together with the attached CO₂ forms a readily hydrolyzable ester group; from an intermediate of the formula

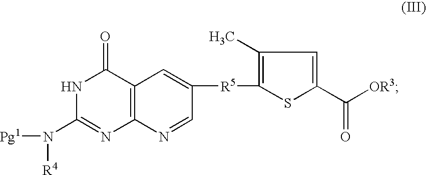

wherein R³ is a moiety that together with the attached CO₂ forms a readily hydrolyzable ester group;

Pg¹ is an amino protecting group;

R⁴ is H;

or Pg¹ can optionally be taken together with R⁴ and the nitrogen to which Pg¹ and R⁴ are attached to form (i) an imine; or (ii) a fused or bridged bicyclic ring or a spirocyclic ring, wherein said ring is saturated and contains from 5 to 12 carbon atoms in which up to 2 carbon atoms are optionally replaced with a hetero moiety selected from O, S(O)ⱼ wherein j is an integer from 0 to 2, and —NR⁸—, provided that two O atoms, two S(O)ⱼ moieties, or an O atom and a S(O)ⱼ moiety are not attached directly to each other;

R⁵ is selected from the group consisting of —C≡C— and —CH═CH—; and

R⁸ is independently H or C₁-C₆ alkyl;

to form the compound of the formula (I) that is optically pure; and to processes for preparing intermediates thereof.

This application claims the benefit of U.S. Provisional Application No. 60/482,320, filed Jun. 25, 2003, the contents of which is hereby incorporated by reference in its entirety.

### BACKGROUND OF THE INVENTION

This invention relates to the novel preparation of a GARFT inhibitor containing a methyl substituted thiophene core and intermediates thereof.

The large class of antiproliferative agents includes antimetabolite compounds. A particular subclass of antimetabolites known as antifolates or antifoles are antagonists of the vitamin folic acid. Typically, antifolates closely resemble the structure of folic acid and incorporate the characteristic para-benzoyl glutamate moiety of folic acid. The glutamate moiety of folic acid takes on a double negative charge at physiological pH. Therefore, this compound and its analogs have an active energy driven transport system to cross the cell membrane and exert a metabolic effect. Glycinamide ribonucleotide formyl transferase (GARFT) is a folate dependent enzyme in the de novo purine biosynthesis pathway. This pathway is critical to cell division and proliferation. Shutting down this pathway is known to have an antiproliferative effect, in particular, an antitumor effect. Thus, a number of folate analogs have been synthesized and studied for their ability to inhibit GARFT. A prototypical specific tight binding inhibitor of GARFT, 5,10-dideazatetrahydrofolic acid, has been reported to show antitumor activity. See F. M. Muggia, “Folate antimetabolites inhibitor to de novo purine synthesis,” New Drugs, Concepts and Results in Cancer Chemotherapy, Kluwer Academic Publishers, Boston (1992), 65-87. One such GARFT inhibitor is:

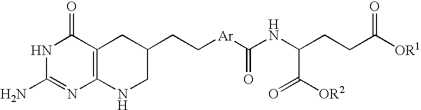

wherein Ar is a substituted or unsubstituted five- or six-membered aromatic group, and wherein each of R¹ and R² are independently a hydrogen atom or a moiety that together with the attached CO₂ forms a readily hydrolyzable ester group. Compounds of formula I are generically described in U.S. Pat. No. 5,646,141, the disclosure of which is incorporated by reference herein. One process of preparing such a GARFT inhibitor is described in U.S. Pat. No. 5,981,748.

An efficient preparation of a GARFT inhibitor wherein Ar is 4-methyl substituted thiophene is desirable.

### SUMMARY OF THE INVENTION

This invention is directed to convergent processes for the preparation of a GARFT inhibitor containing a methyl substituted thiophene core having the following structure:

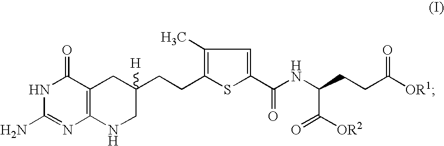

wherein each of R¹ and R² are independently a hydrogen atom or a moiety that together with the attached CO₂ forms a readily hydrolyzable ester group; wherein the method comprises the following steps:

(a) reacting a compound of the formula (III):

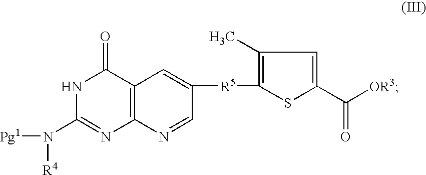

wherein R³ is a moiety that together with the attached CO₂ forms a readily hydrolyzable ester group;

R⁴ is H;

Pg¹ is amino protecting group; or

Pg¹ can optionally be taken together with R⁴ and the nitrogen to which Pg¹ and R⁴ are attached to form (i) an imine; or (ii) a fused or bridged bicyclic ring or a spirocyclic ring, wherein said ring is saturated and contains from 5 to 12 carbon atoms in which up to 2 carbon atoms are optionally replaced with a hetero moiety selected from O, S(O)ⱼ wherein j is an integer from 0 to 2, and —NR⁸—, provided that two O atoms, two S(O)ⱼ moieties, or an O atom and a S(O)ⱼ moiety are not attached directly to each other;

R ⁵ is selected from the group consisting of —C≡C— and —CH═CH—; and

R⁸ is independently H or C₁-C₆ alkyl;

with a hydrogenating agent in the presence of a transition-metal catalyst; preferably said catalyst contains one or more metals selected from the group consisting of palladium and platinum, more preferably palladium;

to form a compound of the formula (IV):

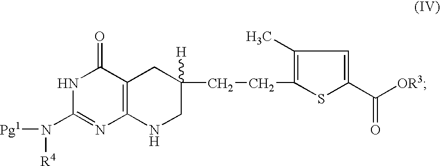

wherein each of said Pg¹, R³, and R⁴ are as described above;

(b) reacting said compound of formula (IV), with a base under an aqueous condition, preferably at an elevated temperature, more preferably at the reflux temperature, to obtain a compound of the formula (II):

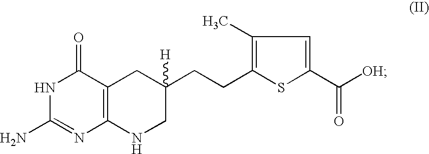

or a salt thereof; and

(c) reacting said compound of the formula (II) with a coupling agent and a base, followed by an L-glutamic acid diester salt, preferably L-glutamic acid di-tert-butyl ester salt, to form said compound of the formula (I).

Suitably, each R¹ and R² are independently C₁-C₆ alkyl, —(CR¹⁰R¹¹)ₜ(C₆-C₁₀ aryl) and —(CR¹⁰R¹¹)^{t}(4-10 membered heterocyclic), wherein t is an integer from 0 to 5; 1 or 2 ring carbon atoms of the heterocyclic group are optionally substituted with an oxo (═O) moiety; and each R¹⁰ and R¹¹ is independently H or C₁-C₆ alkyl. Preferably, each R¹ and R² are independently C₁-C₆ alkyl or benzyl. More preferably, each R¹ and R² are independently methyl, ethyl or tert-butyl.

Suitably, each R³ is C₁-C₆ alkyl, —(CR¹⁰R¹¹)ₜ(C₆-C₁₀ aryl) and —(CR¹⁰R¹¹)ₜ(4-10 membered heterocyclic), wherein t is an integer from 0 to 5; 1 or 2 ring carbon atoms of the heterocyclic group are optionally substituted with an oxo (═O) moiety; and each R¹⁰ and R¹¹ is independently H or C₁-C₆ alkyl. Preferably, each R³ is C₁-C₆ alkyl or benzyl. More preferably, each R³ is methyl or ethyl.

Suitably, each Pg¹ is an amino protecting group selected from the group consisting of trichloroethoxycarbonyl, benzyloxycarbonyl (Cbz), chloroacetyl, trifluoroacetyl, phenylacetyl, formyl, acetyl, benzoyl, tert-butoxycarbonyl (Boc), para-methoxybenzyloxycarbonyl, diphenylmethoxycarbonyl, phthaloyl, succinyl, benzyl, diphenylmethyl, triphenylmethyl (trityl), methanesulfonyl, para-toluenesulfonyl, pivaloyl, trimethylsilyl, triethylsilyl, triphenylsilyl, and the like. Preferred Pg¹ amino protecting group of this invention is (CH₃)₃C—(C═O)—.

Preferably, said compound of formula II is a salt of the following formula (IIa):

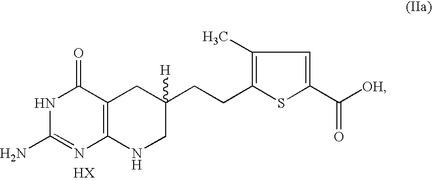

wherein X is halo, preferably chloro.

In one aspect of the invention, said step (c) comprises the following steps:

(c-1) reacting said compound of the formula (II) with a coupling agent and a base, followed by an L-glutamic acid diester salt, to form a diastereomeric mixture of compounds of the formula (Ib):

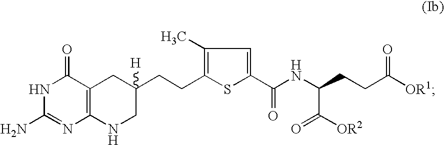

or salts thereof, wherein each of R¹ and R² are as described above;

(c-2) separating through a separation means said diastereomeric mixture of compounds of the formula (Ib) into compounds (Ic) and (Id):

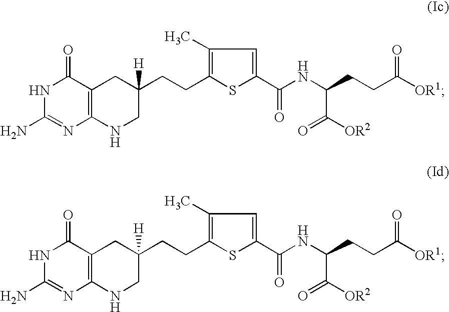

wherein each of R¹ and R² are as described above; and

(c-3) reacting said compound (Ic) with a suitable deprotecting agent to form an optically active compound or salt of the formula (Ia):

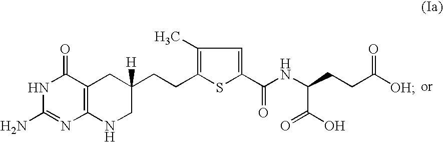

(c-4) reacting said compound (Id) with a suitable deprotecting agent to form an optically active compound or salt of the formula (Ie):

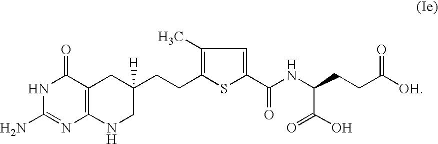

In step (c-1), suitably said coupling agents include any agents able to facilitate formation of an amide bond, such as those compounds forming an activated oxazoline ester (for example, 1-hydroxybenzotriazole or N-hydroxysuccinimide) or anhydride (for example, an acid chloride such as pivaloyl chloride or bis(2-oxo-3-oxazolidinyl)-phosphinic chloride), particularly coupling agents comprising a compound such as a carbodiimide (e.g., dicyclohexylcarbodiimide (DCC), 1,3-diisopropylcarbodiimide (DIC), or 1-(3-dimethylaminopropyl)-3-ethyl-carbodiimide hydrochloride), bis(2-oxo-3-oxazolidinyl)phosphinic chloride), N,N-carbonyl diimidazole (CDI), chloro dimethoxy 1,3,5-triazine (CDMT), pivaloyl chloride, or 2,4,6-trichlorobenzoyl chloride; wherein the aforementioned compounds are preferably employed together with a compound such as 1-hydroxybenzotriazole or N-hydroxysuccinimide, or an amine such as triethylamine, imidazole, pyridine or pyridine substituted at the 4-position with an amine (to form a compound such as 4-dimethylaminopyridine (DMAP) or cyclic amine (to form a compound such as 4-morpholinopyridine or 4-pyrrolidinopyridine). Preferably, said coupling agent is N,N-carbonyldiimidazole (CDI) or chloro dimethoxy 1,3,5-triazine (CDMT).

Preferably, in step (c-1), the base is an amine base, more preferably imidazole.

Preferably said L-glutamic acid diester salt is L-glutamic acid diester hydrochloride, more preferably glutamic acid di-tert-butylester hydrochloride.

Said separation means include any conventional chromatography, derivatization, crystallization, or enzyme separation techiniques; preferably chromatography; more preferably chiral stationary phase chromatography on ChiralPak AD preparatory column, in a solvent, preferably 1:1 heptane:isopropanol mobile phase.

Said suitable deprotecting agents include an acid (such as aqueous sulfuric acid, aqueous hydrogen halide solution (such as HCl), methane sulfonic acid, trifluoroacetic acid or phosphoric acid), a base (such as hydroxide ion or a combination of agents producing a hydroxide base in situ), enzymes (such as esterases, hydrolases or lipases), or hydrogenolysis (such as hydrogen gas in the presence of metal catalyst); preferably the deprotecting agent is an acid; more preferably aqueous sulfuric acid.

In another embodiment of the method of the invention, prior to reacting with a deprotecting agent, the separated compounds (Ic) and (Id) can be independently further purified through a purification means, including purification by column chromatography including Reverse Phase or Normal Phase column chromatography in a solvent, preferably Reverse Phase column chromatography using mixtures of acetonitrile and water as a solvent, wherein the water phase may contain salts such as phosphate salts, or other additives, such as an acid, or a combination thereof.

In another embodiment, the method of the invention further comprises the following steps of preparing said compound of formula (III):

(d-1) reacting a compound of the formula (Va):

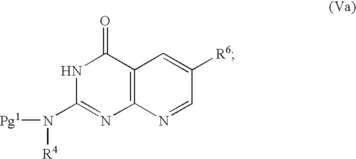

wherein R⁶ is halo, triflate or other activating group;

with a compound of formula (VIb), in the presence of a catalyst, a base, and a solvent,

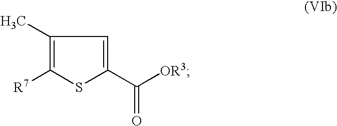

wherein R⁷ is —C≡CH; and R³ is a moiety that together with the attached CO₂ forms a readily hydrolyzable ester group.

Preferably R⁶ in step (d-1) is halo, more preferably bromo.

Preferably said catalyst in step (d-1) contains palladium, more preferably Pd(—O—(C═O)—CH₃)₂ in the presence of a ligand, preferably triphenyl phosphine.

Preferably said base in step (d-1) is an amine, more preferably triethylamine.

Preferably said solvent in step (d-1) is a polar aprotic solvent, more preferably acetonitrile.

Alternatively, in another embodiment, the method further comprises the following steps of preparing said compound of formula (III):

(d-2) reacting a compound of the formula (Va), as described above:

with a compound of formula (VIa), in the presence of a catalyst, a base, and a solvent:

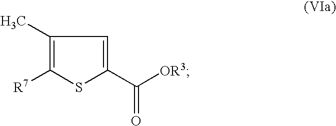

wherein R⁷ is —CH═CH₂; and R³is a moiety that together with the attached CO₂ forms a readily hydrolyzable ester group.

Preferably R⁶ in step (d-2) is halo, more preferably bromo.

Preferably said catalyst in step (d-2) contains palladiums, more preferably Pd(—O—(C═O)—CH₃)₂ in the presence of tri-o-tolyl phosphine.

Preferably said base in step (d-2) is an amine, more preferably diisopropylamine.

Preferably said solvent in step (d-2) is a polar aprotic solvent, more preferably acetonitrile, heated at 85° C.

Yet alternatively, in another embodiment, the method further comprises the following steps of preparing said compound of formula (III):

(d-3) reacting a compound of the formula (Vb):

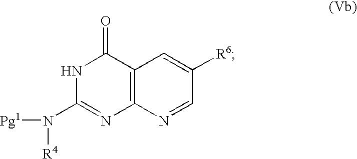

wherein R⁶ is —C≡CH, and Pg¹ and R⁴ are as described above;

with a compound of formula (VIc), in the presence of a catalyst, a base, and a solvent:

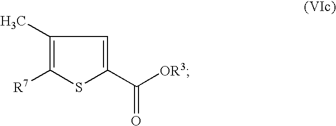

wherein R⁷ is halo, triflate or other activating group; and R³ is as described above.

Preferably said catalyst in step (d-3) contains palladium, preferably the catalyst is palladium halide, most preferably the catalyst is PdCl₂((C₆H₅)₃P)₂, in the presence of a ligand, preferably phosphine ligands. Additionally, a transition metal halide, preferably copper halide, such as Cul, can be used.

Preferably said base in step (d-3) is an amine, more preferably triethylamine.

Preferably said solvent in step (d-3) is a polar aprotic solvent, more preferably acetonitrile, heated at 50° C.

Yet alternatively, in another embodiment, the method further comprises the following steps of preparing said compound of formula (III):

(d-4) reacting a compound of the formula (Vc):

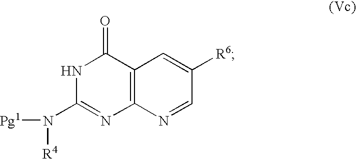

wherein R⁶ is —CH═CH₂, and Pg¹ and R⁴ are as described above;

with a compound of formula (VIc), as described above, in the presence of a catalyst, a base, and a solvent.

Preferably said catalyst in step (d-4) contains palladium, preferably the catalyst is palladium acetate, in the presence of a ligand, preferably phosphine ligands.

Preferably said base in step (d-4) is an amine, more preferably diisopropylamine.

Preferably said solvent in step (d-4) is a polar aprotic solvent, more preferably butyronitrile, heated at 115° C.

In another embodiment, the method of the invention further comprises the following steps of preparing said compound of formula (Vb):

(e-1) reacting said compound of formula (Va), as described above, with a reagent having a formula H—C≡C-Pg², to form a compound of the formula

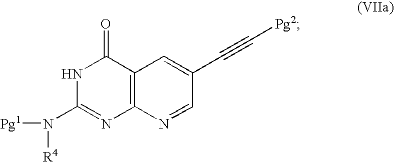

wherein Pg² is a protecting group; and

(f) reacting said compound of formula (VIIa) with a deprotecting agent in a solvent to obtain said compound of the formula (Vb).

Preferably, step (e-1) is performed in the presence of a catalyst, a base and a solvent.

Preferably said catalyst used in step (e-1) is palladium halide, most preferably the catalyst is PdCl₂((C₆H₅)₃P)₂, in the presence of a ligand, preferably phosphine ligands.

Preferably said base used in step (e-1) is selected from the group consisting of hydroxides, carbonates, hydrides, and amines. More preferably said base is an amine, most preferably triethylamine.

Preferably said solvent used in step (e-1) is acetonitrile.

Preferably in step (e-1), Pg² is trimethylsilyl.

Preferably said deprotecting agent used in step (e is carbonate base.

Preferably said solvent used in step (f) is alcohol, more preferably methanol.

Preferably said step (f) is followed by an aqueous acid work-up, more preferably diluted aqueous HCl, and filtration.

Suitably in step (e-1), said reagent of formula H—C≡C-Pg² is selected from the group consisting of:

(i) H—C≡C—Si(R⁹)₃, wherein R⁹ is selected from the group consisting of C₁-C₆ alkyl, —(CR¹⁰R¹¹)ₜ(C₆-C₁₀ aryl) and —(CR¹⁰R¹¹)ₜ₍4-10 membered heterocyclic), wherein t is an integer from 0 to 5; 1 or 2 ring carbon atoms of the heterocyclic group are optionally substituted with an oxo (═O) moiety; and each R¹⁰ and R¹¹ is independently H or C₁-C₆ alkyl;

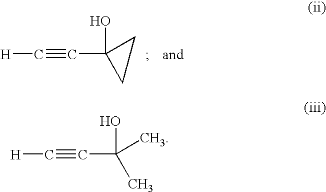

Preferably said reagent of the formula H—C≡C-Pg² is selected from the group consisting of

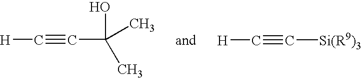

Preferably R⁹ is C₁-C₆ alkyl, more preferably methyl.

In another embodiment, the method of the invention further comprises the following steps of preparing said compound of formula (Vc):

(e-2) reacting said compound of formula (Va), as described above,

with a reagent having a formula CH₂═CH₂, in the presence of a base, catalyst and solvent, to form said compound of the formula (Vc).

Preferably said base used in step (e-2) is selected from the group consisting of hydroxides, carbonates, hydrides, and amines. More preferably said base is an amine, most preferably triethylamine.

Preferably said catalyst used in step (e-2) is palladium acetate, in the presence of a ligand, preferably phosphine ligands. Said ligands include tri-o-tolyl phosphine and BINAP, or a combination thereof.

Preferably said solvent used in step (e-2) is acetonitrile.

The present invention further relates to a compound of formula (Ib) or a salt thereof:

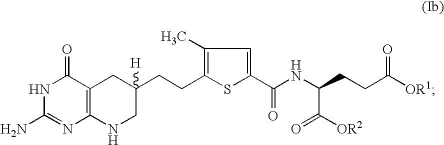

wherein each of R¹ and R² are independently a moiety that together with the attached CO₂ forms a readily hydrolyzable ester group.

The present invention further relates to a compound of formula (III) or a salt thereof:

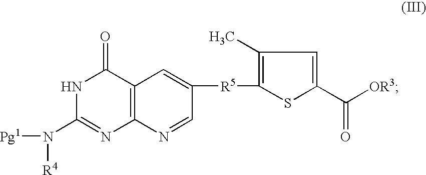

wherein R³ is a moiety that together with the attached CO₂ forms a readily hydrolyzable ester group;

Pg¹ is an amino protecting group;

R⁴ is H; or

Pg¹ can optionally be taken together with R⁴ and the nitrogen to which Pg¹ and R⁴ are attached to form (i) an imine; or (ii) a fused or bridged bicyclic ring or a spirocyclic ring, wherein said ring is saturated and contains from 5 to 12 carbon atoms in which up to 2 carbon atoms are optionally replaced with a hetero moiety selected from O, S(O)ⱼ wherein j is an integer from 0 to 2, and —NR⁸—, provided that two O atoms, two S(O)ⱼ moieties, or an O atom and a S(O)ⱼ moiety are not attached directly to each other;

R⁵ is selected from the group consisting of —C≡C— and —CH═CH—; and

R⁸ is independently H or C₁-C₆ alkyl.

The present invention further relates to a compound of formula (IV), a salt thereof, an enantiomeric mixture thereof, or pure enantiomers thereof:

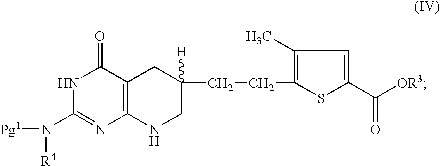

R³ is a moiety that together with the attached CO₂ forms a readily hydrolyzable ester group;

R⁴ is H;

Pg¹ is amino protecting group; or

Pg¹ can optionally be taken together with R⁴ and the nitrogen to which Pg¹ and R⁴ are attached to form (i) an imine; or (ii) a fused or bridged bicyclic ring or a spirocyclic ring, wherein said ring is saturated and contains from 5 to 12 carbon atoms in which up to 2 carbon atoms are optionally replaced with a hetero moiety selected from O, S(O)ⱼ wherein j is an integer from 0 to 2, and —NR⁸—, provided that two O atoms, two S(O)ⱼ moieties, or an O atom and a S(O)ⱼ moiety are not attached directly to each other; and R⁸ is independently H or C₁-C₆ alkyl.

### Definitions

For purposes of the present invention, as described and claimed herein, the following terms are defined as follows:

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight or branched moieties.

The term “aryl”, as used herein, unless otherwise indicated, includes an organic radical derived from an aromatic hydrocarbon by removal of one hydrogen, such as phenyl or naphthyl.

The term “amino protecting group” as used herein, unless otherwise indicated, refer to selectively introducible and removable groups which protect amino groups against undesirable side reactions during synthetic procedures. Examples of amino protecting groups include trichloroethoxycarbonyl, benzyloxycarbonyl (Cbz), chloroacetyl, trifluoroacetyl, phenylacetyl, formyl, acetyl, benzoyl, tert-butoxycarbonyl (Boc), para-methoxybenzyloxycarbonyl, diphenylmethoxycarbonyl, phthaloyl, succinyl, benzyl, diphenylmethyl, triphenylmethyl (trityl), methanesulfonyl, para-toluenesulfonyl, pivaloyl, trimethylsilyl, triethylsilyl, triphenylsilyl, and the like.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following:

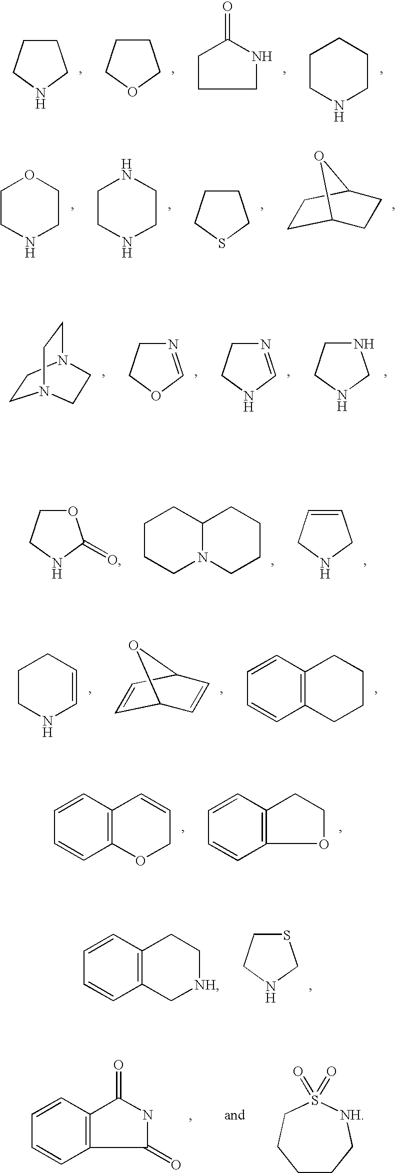

Unless otherwise indicated, the term “oxo” refers to ═O.

Certain compounds of formula (I) may have asymmetric centers and therefore exist in different enantiomeric forms. All optical isomers and stereoisomers of the compounds of formula (I), and mixtures thereof, are considered to be within the scope of the invention. With respect to the compounds of formula (I), the invention includes the use of a racemate, one or more enantiomeric forms, one or more diastereomeric forms, or mixtures thereof. The compounds of formula (I) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patni et al., Chem. Rev, 1996, 96, 3147-3176 and references cited therein.

The subject invention also includes isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, 35S, 18F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples and Preparations below, by substituting a readily available isotopically labelled reagent for a non-isotopically labelled reagent.

Other aspects, advantages, and preferred features of the invention will become apparent from the detailed description below.

### DETAILED DESCRIPTION OF THE INVENTION AND ITS PREFERRED EMBODIMENTS

Acccording to a process of this invention, compound of formula I can be prepared by the reaction schemes depicted below:

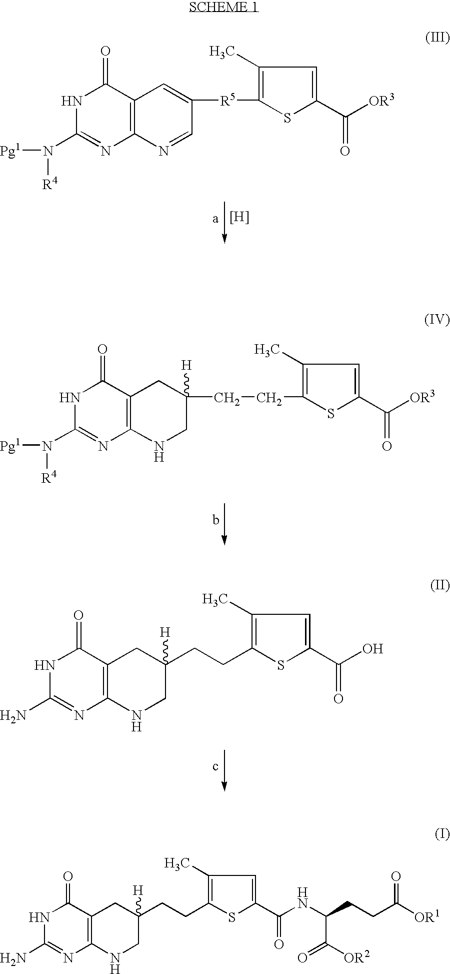

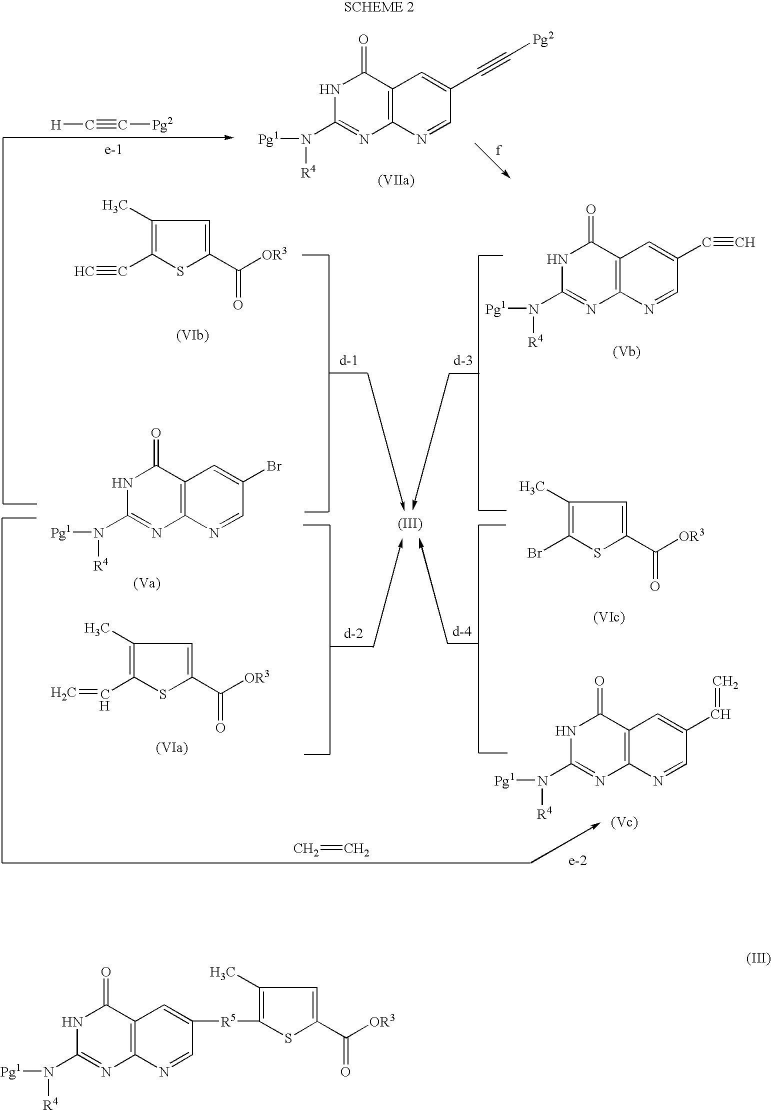

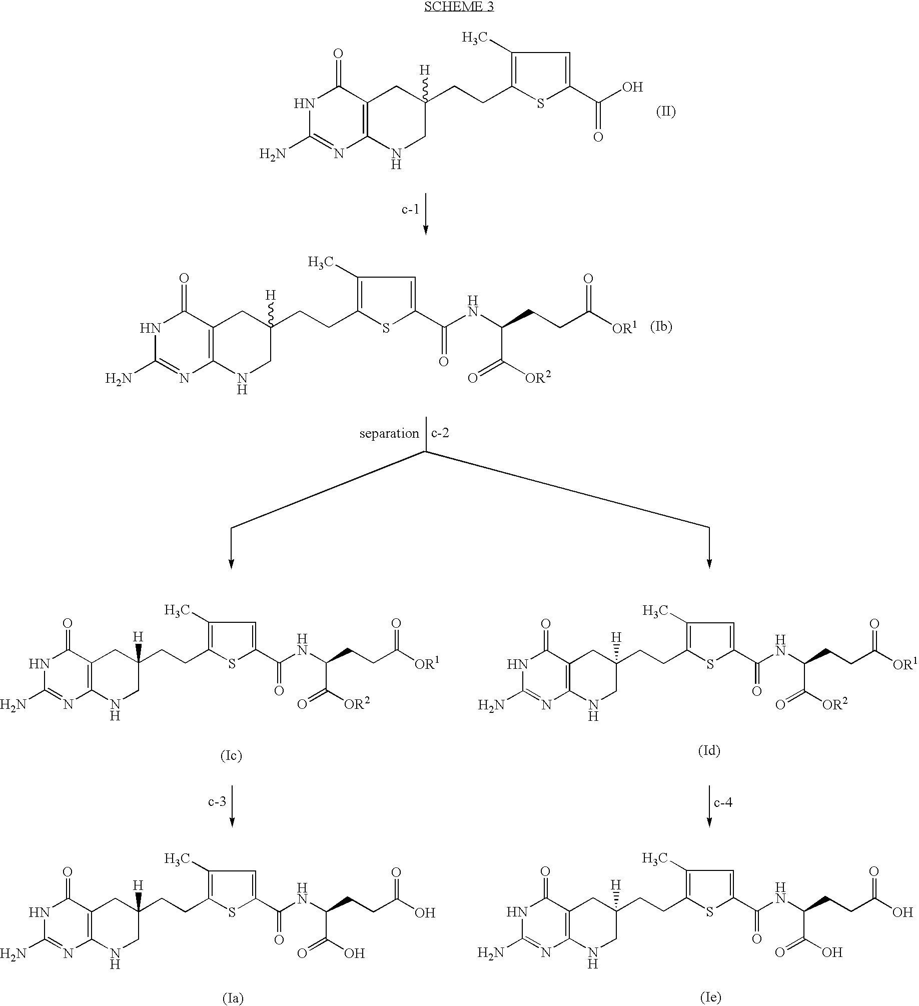

### Description of Schemes

Scheme 1 illustrates the preparation of compounds of formula (I). According to Scheme 1, a compound of formula (I) can generally be prepared as follows from a compound of formula (III). A compound of formula (III) can be treated with hydrogen under high pressure in the presence of a palladium catalyst to afford compound (IV). Said compound (IV) is then saponified under reflux with a base such as aqueous sodium hydroxide, which, upon acidification with aqueous hydrochloric acid, affords compound (II). The carboxylate functionality of compound (II) is activated in the prescence of a base, subsequently coupled with a glutamic acid diester salt, and finally deprotected to provide a compound of formula (I).

Scheme 2 illustrates the preparation of compounds of formula (III), which can be used as a starting material in Scheme 1. According to Scheme 2, a compound of formula (III) can generally be prepared as follows by a palladium-catalyzed reaction of a compound of formula (V), i.e., compound of formula (Va), (Vb), or (Vc) respectively, with a compound of formula (VI), i.e., compound of formula (VIa), (VIb), or (VIc) respectively.

Compounds of formula (Vc) can be prepared from compounds of formula (Va) by reacting said compounds of formula (Va) with ethylene in a palladium-catalyzed reaction according to step (e-2). Compounds of formula (Vb) can be prepared from compounds of formula (Va) by reacting said compound of formula (Va) with a mono-protected acetylene in the presence of a base under elevated temperature in a palladium-catalyzed coupling reaction according to step (e-1), followed by a subsequent base-induced cleavage of the acetylene protecting group according to step (f).

Under conditions similar to steps (e-1) and (e-2) compounds of formula (VIa) and (VIb) can be prepared from compounds of formula (VIc) via a palladium-catalyzed coupling reaction with ethylene or acetylene (preferably mono-protected), in the presence of a base at elevated temperature.

Scheme 3 illustrates the preparation of compounds of formula (Ia) and (Ie), respectively. According to Scheme 3, in step (c-1), the carboxylate functionality of a racemic mixture of a compound of formula (II) is activated, for example by treatment with 1,1-carbonyl diimidazole, and subsequently coupled with a glutamic acid diester salt, such as di-O-t-butyl glutamate hydrochloride in N-methylpyrrolidinone, affording a compound of formula (Ib) consisting of a mixture of two diastereomeric compounds of formula (Ic) and (Id). Said two diastereomers (Ic) and (Id) are then separated by means of chiral stationary phase column chromatography and further purified by column chromatography according to step (c-2). In step (c-3) and (c-4), a compound of formula (I), i.e. a compound of formula (Ia) or (Ie), respectively, can generally be prepared by treatment of a compound of formula (Ic) and (Id), respectively, with an acid, followed by basic aqueous workup and re-acidification to form deprotected (Ia) and (Ie), respectively.

Compounds of formula (Va) can be prepared by the methods described in: (a) Taylor, Edward C.; Wong, George S. K. Joumal of Organic Chemistry 1989, 54, 3618-3624. (b) Taylor, Edward C.; Wong, George S. K. Eur. Pat. Appl. 1988, EPXXDW EP 265126 A2.

Compounds of formula (Vb) can be prepared by the methods described in: Taylor, Edward C.; Yoon, Cheol Min. Synthetic Communications 1988, 18(11), 1187-1191.

Compounds of formula (Via) can be prepared by the methods described in: (a) Varney, Michael D.; Palmer, Cindy L.; Romines, William H., III; Boritzki, Theodore; Margosiak, Stephen A.; Almassy, Robert; Janson, Cheryl A.; Bartlett, Charlotte; Howland, Eleanor J.; Ferre, Rosanne Journal of Medicinal Chemistry 1997, 40, 2502-2524. (b) Varney, Michael D.; Romines, William H.; Palmer, Cynthia L. PCT Int. Appl. 1996, WO 9640674.

### EXAMPLES

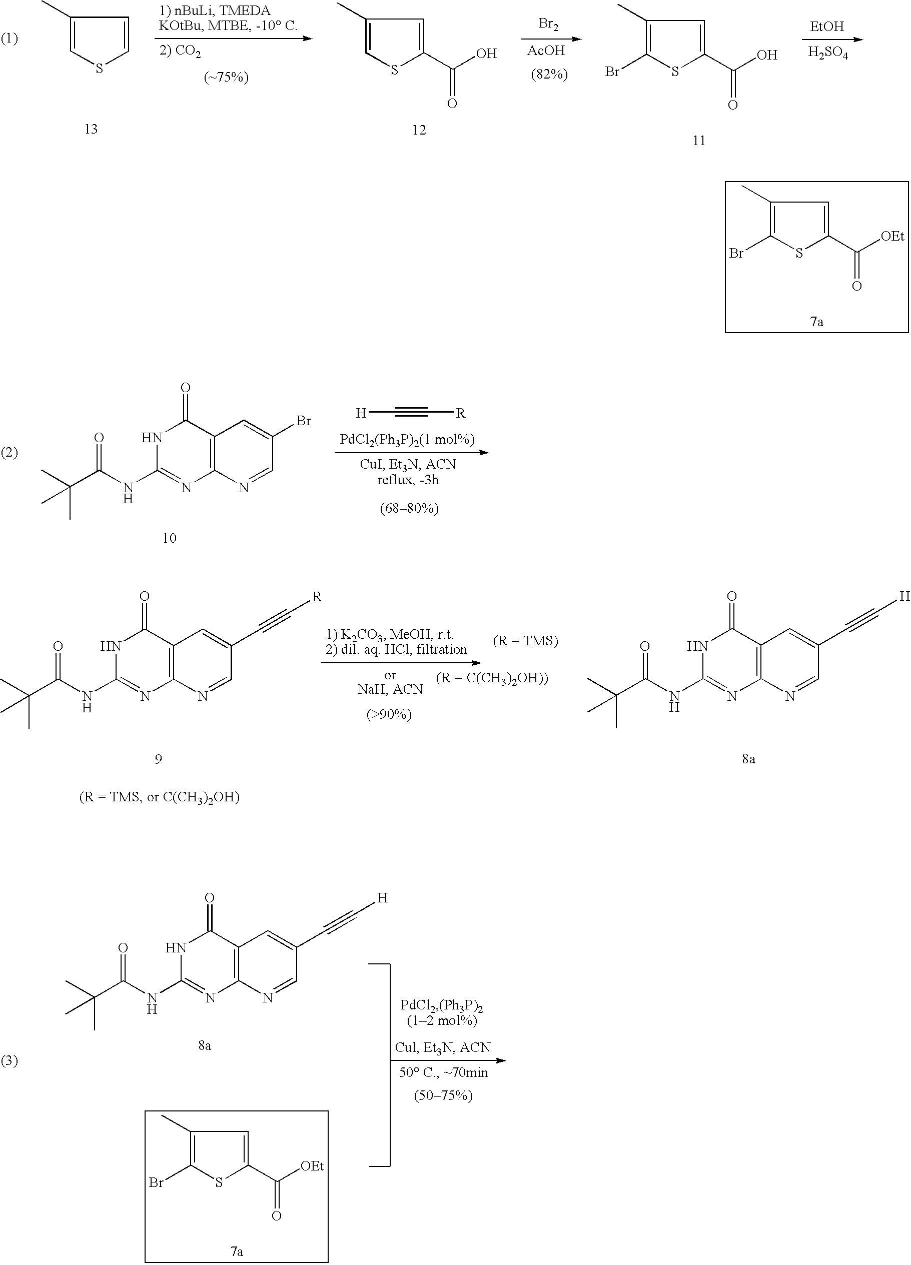

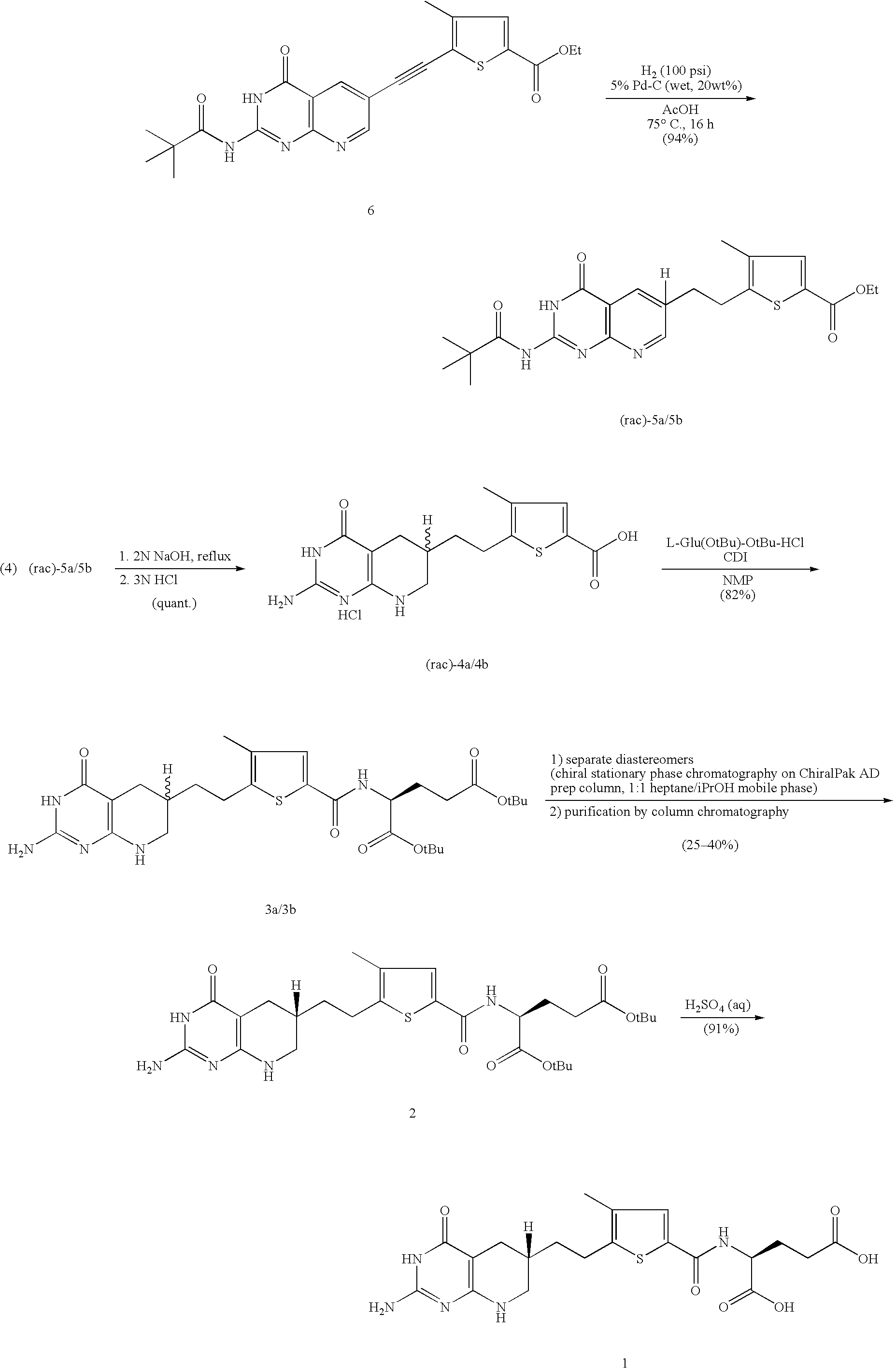

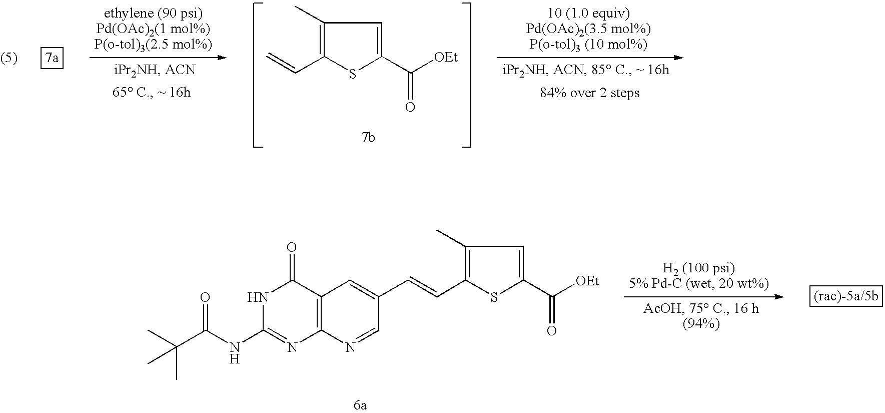

Preparation of Compound 7a

TMEDA (99.5%), 3-methylthiophene (98%) and KOtBu (95%) were obtained from Aldrich and used without further purification. 2.5M n-BuLi in hexanes was obtained from Aldrich and Acros and the exact molarity of each lot was determined via titration prior to use. MTBE used was from EMScience (spectrophotometric grade). A 50 L glass reactor equipped with overhead stirrer and connected to a chiller was purged with nitrogen (20 min.). To the reactor was charged MTBE 20 (20.8 L), TMEDA (2.08 L) and KOtBu (94.8 g). The reactor was closed and the stirred mixture was cooled down to −8° C. under a steady stream of nitrogen. The temperature was monitored via thermocouple and chart recorder. To the reactor was slowly charged a pre-cooled 2.5 M solution of n-BuLi in hexanes (3.38 L) from an addition funnel. The reaction temperature was kept below −5° C. during the addition. Following complete addition, the reaction was stirred an additional 3 hours at −8° C. 3-Methylthiophene 13 (830 g) was slowly charged to the reactor via an addition funnel. Following complete addition, the reaction was stirred an additional 1 hour at −8° C. Dry carbon dioxide gas was introduced into the reaction mixture for 1 hour while keeping the reactor temperature below +15° C. Deionized water (12 L) was carefully added to the reactor with continued stirring. Following complete addition, the chiller was removed and the biphasic mixture was agitated vigorously via overhead stirring. After separation of layers, the lower aqueous layer was drained and set aside. 1N NaOH (5 L) was added to the reactor and the biphasic mixture was agitated vigorously via overhead stirring. After separation of layers, the lower aqueous layer was drained and set aside. This operation was repeated one more time with 1N NaOH (10 L). After the last aqueous phase had been drained from the reactor and set aside, the remaining upper organic layer was drained and discarded. The reactor was given a quick rinse with water and acetone and was allowed to dry. All aqueous layers were recharged to the reactor and stirring was resumed. Concentrated HCl (˜4 L) was added via addition funnel until pH 1-2 was achieved. MTBE (10 L) was charged to the reactor and the biphasic mixture was agitated vigorously via overhead stirring. After separation of layers, the upper organic phase layer was transferred via vacuum line from the reactor to a separate receptacle. This operation was repeated two more times with 5 L portions each of MTBE. The combined MTBE layers were dried over MgSO4, filtered, and the solvents were removed under reduced pressure to give the product as a grayish solid. The product was dried in a vacuum oven overnight at 30° C. The dried product was milled to a free-flowing powder (if necessary) to afford 4-methylthiophene-2-carboxylic acid 12 as a lavender-colored solid ( 85% yield, 95:5 ratio of regioisomers).

4-Methylthiophene-2-carboxylic acid 12 (541 g) was dissolved in 4.59 L glacial acetic acid and then cooled to an internal temperature of 10-15° C. Then, a solution of bromine in acetic acid (prepared by dissolving 195 mL of bromine in 2.8 L glacial acetic acid) was added over 1.5 hours while stirring vigorously to the vigorously stirred solution of 2a/2b in acetic acid. After 30 minutes, the reaction mixture is quenched over 30-40 minutes into 19 L of water (at room temperature) with vigorous stirring providing a fine precipitate. The resulting precipitate was collected by vacuum filtration (over a couple of hours) and rinsed with 2 L of water. As much of the excess water as possible was pressed from the filter cake by placing a latex rubber dam over the stainless steel, lowform Buchner funnel for 2-3 hours. The resulting filter cake, still containing about 600 mL of water, was then broken up into chunks in a Pyrex dish/tray and dried in vacuo (with a slight air bleed) at 65-70° C. for 1.5-3 days. This afforded 5-bromo-3-methylthiophene-2-carboxylic acid 11 (692 g, 82% yield) as single regioisomer (determined by 1H NMR) as a cement-gray solid.

5-Bromo-4-methylthiophene-2-carboxylic acid 11 (690 g) and absolute ethanol (7.4 L) were combined and stirred. Once all of the solids had dissolved, concentrated sulfuric acid (270 mL) was added carefully. The solution was then heated to gentle reflux, under a calcium sulfate drying tube or slight positive pressure of nitrogen (with a bubbler), for 18 hours. The reaction was monitored for disappearance of starting material by 1H NMR or other appropriate analytical method. In the case of an incomplete reaction, a further 0.2 to 0.3 equivalents of concentrated sulfuric acid and 1 L of absolute ethanol was added to the reaction and reflux was continued for another 3 to 4 hours. Upon complete reaction, the reaction was cooled to room temperature and carefully quenched, to a pH of 8, by portion-wise addition of an excess of solid sodium bicarbonate to the vigorously stirred solution (initial induction period between the addition of the solid and the evolution of carbon dioxide!). The resulting slurry was concentrated in vacuo to remove most of the ethanol. Enough water was then added to dissolve the salts (sodium bicarbonate, sodium sulfate, etc.) and this mixture was extracted twice with 4 L portions of MTBE. The MTBE extracts were combined and washed twice with 4 L portions of saturated aqueous sodium bicarbonate. The organic phase was concentrated in vacuo to give ethyl 5-bromo-4-methyl-thiophene-2-carboxylate 7a (726 g, 2.92 moles, 83% yield) as dark colored oil.

Preparation of Compound 9:

A 3 L Morton flask equipped with mechanical stirrer, temperature probe, reflux condenser and Ar inlet was charged with of 10 (100 g, 307.5 mmol), PdCl₂(PPh₃)₂ (4.32 g, 6.15 mmol, 0.02 equiv), Cul (1.17 g, 6.15 mmol, 0.02 equiv) and degassed acetonitrile (500 mL, 5 vol/wt). The resulting slurry was sparged with Argon for 1 hour while stirring at room temperature. The mixture was charged with degassed triethylamine (128 mL, 923 mmol, 3 equiv) and sparged with Argon for an additional 15 minutes at room temperature. The resulting bright yellow slurry was charged with trimethylsilylacetylene (87 mL, 615 mmol, 2 equiv) then heated to 70° C. while stirring rapidly under inert atmosphere. When HPLC indicated no more consumption of 10 (3 to 4 hours), the mixture was cooled to 0° C. and filtered. The resulting yellow cake was re-slurried in 50% aqueous acetonitrile (0.5 L) at 0° C., stirred for 0.5 h, then filtered. The product was then washed with cold acetonitrile (0.5 L) and allowed to dry overnight in a vacuum oven at 50 ° C. Desired product 9 was obtained as a light yellow to off-white solid, 92.17 g (87.5%) contaminated with 6.6% of unreacted 10. 1H NMR(CDCl3, 250 MHz) δ 0.29 (s, 9H), 1.35 (s, 9H), 8.36 (br s,1H), 8.57 (d, 1H, J=2.45 Hz), 8.92 (d, 1H, J=2.45 Hz). This material was taken on to the deprotection step without further purification.

Compound of the formula 10 may be prepared by methods known to those skilled in the art. For example, see: Taylor, Edward C.; Yoon, Cheol Min. Synthetic Communications 1988, 18(11), 1187-1191.

Preparation of Compound 8a:

An off-white suspension of 9 (92 g, 269 mmol) in MeOH (1380 mL,15 vol/wt) was cooled to 0° C., charged with K₂CO₃ (56 g, 403 mmol, 1.5 equiv) and stirred vigorously at 0° C. for 1-2 hours until HPLC indicated complete consumption of 9. While keeping internal temperature below 5° C. the mixture was quenched to pH 2 using 1N HCl (806 mL) and diluted with 574 mL cold water. After stirring at 0° C. for 1 hour, the suspension was filtered and the resulting solid washed with 1 L cold water. Drying for 16 hours in a 50° C. vacuum oven yielded desired product 8a as an off-white solid, 77.05 g (106%). HPLC revealed this material to consist of 8a (83%) and 10 (12%). 1H NMR(CDCl_{3,} 250 MHz) δ 1.36 (s, 9H), 3.31 (s, 1H), 8.39 (brs, 1H), 8.60 (d, 1H, J=1.99 Hz), 8.49 (d, 1H, J=1.99 Hz). This material was taken directly to the next step without purification.

Preparation of Compound 6:

Into a 3 L Morton flask equipped with a mechanical stirrer, condenser, temperature probe and Ar inlet was charged 8a (77.05 g, 269 mmol), 7a (73.61 g, 296 mmol, 1.2 equiv), PdCl₂(PPh₃)₂ (3.77 g, 5.37 mmol, 0.02 equiv), Cul (1.023 g, 5.37 mmol, 0.02 equiv) and degassed acetonitrile (1 L, 10 vol/wt). The yellow suspension was sparged with Argon for 1 h then charged with degassed triethylamine (112 mL, 806 mmol, 3 equiv). The resulting mixture was heated at 50° C. for 70 minutes while stirring vigorously. When the reaction was complete as indicated by HPLC, the mixture was cooled to 0° C. and quenched with 1 N citric acid (806 mL, 3 equiv). After 1 hour at 0° C., the suspension was filtered and the resulting product cake was washed successively with 1 L cold water, 1 L cold 50% aqueous acetonitrile and 1 L cold acetonitrile. After drying in a 50° C. vacuum oven, the desired product 6 (92.8 g, 79% yield) was obtained as a yellow solid. The product was further purified by dissolving in DMSO (1860 mL, 20 vol/wt) at 85° C. and allowing to crystallize slowly as the dark solution was cooled gradually to rt. The resulting light yellow solid was filtered, washed with two 1 L-portions of water then with 0.5 L cold acetonitrile. After drying overnight in a 50° C. vacuum oven, pure 6 was obtained in 77% yield.

Hydrogenation of 6-Synthesis of Racemic 5a/5b

A 19.5 L Stirred Parr Reactor is charged with a slurry of 6 (610.0 g, 1.377 mol), or alternatively 6a, in acetic acid (2.5 L), followed by a slurry of 5% Pd/C (122.0 g, 1.146 mol, 50% wet, Johnson-Matthey Type A 102023-5, JM #078622008) in acetic acid (800 mL). This mixture is then diluted with additional acetic acid (3.9 L), which, if necessary, can be used to rinse any residual substrate and catalyst into the reactor. Then the reactor is closed in preparation for introduction of hydrogen gas and start of the reaction. For that purpose, the reactor is purged with three cycles of nitrogen charges (˜50 psi each) in order to purge air from the reactor. Following the nitrogen purges, the reactor is flushed with hydrogen (3×˜50 psi), and then charged with hydrogen at 100 psi hydrogen gas. The reaction mixture is slowly heated to 75° C. so as not to overshoot setpoint temperature by too much, and agitated at 700 rpm. The reaction is held overnight (˜16 h) at 75° C. and 100 psi in order to ensure that levels of partially reduced intermediates are minimized. For minimal reaction time and best results, it is essential to recharge hydrogen gas as necessary to keep reactor pressure around 100 psi, as hydrogen is rapidly consumed in the early stages of the reaction. Monitoring by HPLC revealed that the reaction is complete after 16 h with conversion rates typically being 97+%. The reactor heating is turned off, the hydrogen gas in the reactor is drained and replaced with an inert atmosphere, and then the reactor is drained and efficiently rinsed with acetic acid (1.25 L). The rinse is combined with the drained and still warm reaction mixture. Next, the catalyst is removed by filtration in as short a time as possible through a short bed of Celite contained in a 2-3 L sintered glass funnel. It is important that the reaction mixture be still warm during the filtration in order to avoid premature crystallization of product from acetic acid upon cooling. The filter cake is washed with warm acetic acid (1.8 L). The resulting filtrate (˜10.25 L) is transferred to a Distillation Reactor and concentrated to low volume (˜1.25 L) at 45 to 65° C. During the distillation of acetic acid, the product will begin to crystallize as a white solid. Then, acetonitrile (10 L) is slowly charged to the concentrated product in acetic acid while continuing agitation. This dilution is accompanied by further crystallization of the product. Next, the mixture is cooled to 4° C. with an icebath for 1.5 h, the white solid is filtered through a filter funnel, and the filter cake is washed with cold acetonitrile (2 L). The filter cake is dried at 50° C. for 16 hr under house vacuum to afford racemic 5a/5b (92% yield, 99% HPLC purity) as a white, crystalline solid. 1H NMR (DMSO-d₆, 700 MHz): 1.21 (s, 9H, tBu); 1.27 (t, J=7.0 Hz, 3H, CH₂CH₃); 1.57 (m,1H, CH₂); 1.63 (m,1H, CH₂); 1.95 (dd, J=9.1 Hz, J=15.4 Hz, 1H, CH₂); 2.15 (s, 3H, CH₃); 2.56 (dd, J=3.5 Hz, J=15.4 Hz, 1H, CH₂); 2.81-2.91 (m, 3H); 3.29 (m, 1H); 4.23 (quart, J=7.0 Hz, 2H, CH₂CH₃); 6.48 (s,1H, NH); 7.54 (s, 1H, CH); 10.58 (s, 1H, NH); 11.2 (s, 1H, NH). 13 C NMR (DMSO-d₆): 13.174, 14.192, 25.260, 25.287, 26.270, 29,974, 33.821, 40.036, 45.031, 60.563, 87.224, 127.942, 134.279, 136.238, 147.008, 148.827, 158.191, 159.414, 161.373, 181.105

Preparation of 6a via Double Heck-Reaction

This reaction was performed in the Argonaut Endeavor system. Compound 7a (0.20 g, 0.80 mmol) was suspended in 4 mL of degassed acetonitrile. The solution was treated with tri-o-tolylphosphine (6.3 mg, 0.021 mmol), palladium acetate (1.8 mg, 0.008 mmol) and diisopropylamine (0.16 g, 0.22 mL, 1.60 mmol). Then, the system was sealed, and the vessel was purged with argon (5×) followed by ethylene (5×). After the vessel was pressurized with ethylene to 30 psi the solution was warmed to 65° C. The ethylene pressure was increased to 90 psi, and the solution was allowed to stir (1000 rpm) under these conditions for 16 hours. The solution was cooled to room temperature and purged with argon (3×). To the mixture was added 10 (0.26 g, 0.80 mmol), tri-o-tolylphosphine (24.9 mg, 0.08 mmol), palladium acetate (6.4 mg, 0.028 mmol) and diisopropylamine (0.16 g, 0.22 mL, 1.60 mmol), and the system was resealed and purged with argon (5×). The solution was warmed to 85° C. and stirred (1000 rpm) under these conditions for another 16 hours. This solution was then allowed to cool to room temperature and the precipitated product was isolated via vacuum filtration. The crude cake was washed with acetonitrile (2×10 mL), water (2×10 mL) and finally with heptane (10 mL) to give 6a as a golden yellow solid (0.29 g, 84% crude yield). 1H NMR (400 MHz, CDCl3): 8.88 (d, J=1.52 Hz, 1 H), 8.56 (d, J=2.27 Hz, 1 H), 7.47 (s, 1 H), 7.30 (d, J=16.17 Hz, 1 H), 7.19 (s, 2H), 6.94 (d, J=16.17 Hz, 1 H), 4.28 (q, J=7.07 Hz, 2 H), 2.28 (s, 3 H), 1.32 (t, J=7.20 Hz, 3 H), 1.28 (s, 9 H).

Saponification of Racemic 5a/5b:

A 500 mL three-necked round bottom flask is equipped with an overhead stirrer, a reflux condenser, and an addition funnel. The flask is placed into an ice/water bath at 0-5° C., then deionized water (100 mL) is charged to the vessel and agitation is started. Sodium hydroxide pellets (8.00 g) are charged to the reaction vessel. While warming to room temperature, the contents are stirred until all solids have dissolved. Racemic 5a/5b (20.00 g, 44.84 mmol) is then charged to the flask resulting in a yellow slurry. The water bath is replaced with a heating mantle and the reaction mixture is heated to reflux (˜100° C.). Keep heating at reflux temperature until all solids are dissolved and HPLC monitoring reveals that the reaction is complete. This is typically the case after 30-60 min of heating. Then, the heating mantle is removed and a 3N HCl (120 mL) solution is added dropwise via addition funnel while the reaction mixture is still warm (approximately 60° C.). The mixture is stirred vigorously. Upon addition of the HCl-solution, the mixture gradually cools down. A white precipitate is formed, which is a thick slurry while pH=4-5. After the addition is complete (mixture has pH<0), the mixture is stirred while reaching room temperature. The precipitate is filtered by suction via Buchner funnel and washed with water (3×150 mL). Alternatively, the precipitate can be washed with MeCNIH₂O-mixtures while gradually increasing the MeCN portion. For example, MeCN/H₂O (4:1, 200 mL), MeCN/H₂O (3:4, 700 mL), MeCN (500 mL). After the washing, the solid is collected and dried in a vacuum oven at 60° C. with an air bleed until no further loss of weight can be observed (˜24 h). This affords racemic 4a/4b.HCl (15.87 g, 95%) as an off-white solid. For the next step, the obtained material must be homogenized and ground as finely as possible.

Preparation of 3a/3b by Incorporation of L-glutamate via CDI-Activation:

A 3 L three-necked round bottom flask is equipped with an overhead stirrer and an addition funnel (1 L). The third neck is left open. The flask is charged with solid and finely ground racemic 4a/4b.HCl (68.62 g, 0.185 mol). Then, NMP (343 mL) is added and agitation is started. While warming to 40° C., the mixture is stirred vigorously until a very fine suspension is obtained (˜30 min). To this suspension, solid CDI (30 g; 1.0 equiv) is added via a funnel through the open neck. Once the CDI was added, there is an induction period of a few minutes, after which a slight exotherm can be observed accompanied with evolution of CO₂. The progress of the reaction is monitored via HPLC. After 10 min, a sample is taken and the conversion of the starting material to the acyl imidazole intermediate is typically >90%. The reaction mixture was a clear solution at this point. After another 10 min, a second portion of CDI (6 g; 0.2 equiv) is added and the mixture is stirred for another 20 min. Monitoring by HPLC revealed that at this point the conversion to the acyl imidazole intermediate is typically 95% or greater. Only a very small amount of starting material (<5%) is left. Then, water (17 mL) is added and the mixture is stirred for 10 min. The addition of water is slightly exothermic. Next, solid di-O-tBu-L-glutamate.HCl (137.2 g, 2.5 equiv) is added, and the mixture is stirred at 40° C. for 5-6 h, after which no acyl imidazole intermediate should be detected by HPLC. It is important that the acyl imidazole intermediate be consumed completely. Upon vigorous stirring, EtOAc (660 mL) is added in one portion to the reaction mixture through the addition funnel, followed by a 3:1-mixture of H₂O/brine (855 mL). The phases are separated and the aqueous phase is extracted with EtOAc (3×150 mL). The combined EtOAc-phases are washed with aqueous 1N HCl (2×150 mL), followed by extraction with half-saturated sodium bicarbonate solution (2×150 mL). Finally, the organic phase is washed with water (2×150 mL) and concentrated in vacuum at 35° C. This affords a crude mixture of diastereomers 3a/3b (76.8 g, 89%) with an apparent purity of ˜91%. The diastereomers are separated by column chromatography (Chiralpak AD, Heptanes/EtOH=1:1), and the desired diastereomer 2 is further purified by column chromatography (SiO₂; 5% MeOH/CH₂Cl₂; Biotage Flash Column 400) affording pure 2 (26.63 g, 25% overall, de>98%). 1H NMR (DMSO-d₆, 700 MHz): 1.38, 1.40 (2 s, 18H, 2×OtBu); 1.51 (m, 1H); 1.58 (m, 1H); 1.65 (m, 1H, CH); 1.83-1.90(m, 2H); 1.99 (m, 1H); 2.13 (s, 3H, CH₃); 2.28-2.32 (m, 2H); 2.49 (m,1H); 3.19 (m,1H); 4.25 (ddd, J=4.9 Hz, J=7.7 Hz, J=12.6 Hz, 1H, CH—N); 5.94 (s, 2H, NH₂); 6.27 (m, 1H, NH); 7.58 (s, 1H, CH); 8.39 (m, 1H, NH); 9.75 (s, 1H, NH) 13C NMR (DMSO-d₆):13.358, 14.059, 20.727, 25.130, 25.360, 25.998, 27.643, 27.712, 30.617, 31.308, 34.335, 45.114, 52.237. 59.720, 79.775, 80.609, 81.730, 131.502, 133.298, 133.959, 144.038, 153.280, 159.532, 161.393, 161.478, 170.981, 171.469.

t-Bu-ester Deprotection of 2-Preparation of 1:

A three-necked round bottom flask is equipped with an overhead stirrer and charged with a mixture of concentrated H₂SO₄ (15 mL) and water (20 mL) and the mixture is cooled to 0° C. Then, solid 2 (10.00 g, 17.37 mmol) is added in four portions (˜2.5 g each) within 10 min upon vigorous stirring. After 30 min, most of the starting material has dissolved and the mixture is kept at 4° C. After stirring for 16-24 h, the cold reaction mixture is added to a cold solution (−9° C.) of sodium hydroxide (30 g) in water (180 mL) via addition funnel within 45 min. During the quench, the reaction mixture is stirred vigorously, and the internal temperature is kept below 5° C. After the quench is complete the reaction mixture is stirred while maintaining the temperature below 5° C. To this clear slightly yellow solution (pH=12-13) is added 3N HCl (˜35 mL) until pH=3-3.5 upon formation of a white precipitate. After the addition of HCl is complete, the mixture is kept stirring for 45 min. Then, the precipitate is filtered via Buchner funnel, thoroughly washed with excess water, and dried by suction at ambient temperature for 24 h affording 1 (7.36 g, 91%) as a white powder.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

What is claimed is:

1. A method of preparing a compound or a salt of the formula (I): 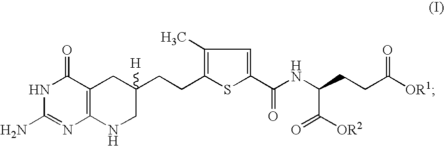
wherein each of R¹ and R² are independently a hydrogen atom or a moiety that together with the attached CO₂ forms a readily hydrolyzable ester group; wherein the method comprises the following steps:
(a) reacting a compound of the formula (III): 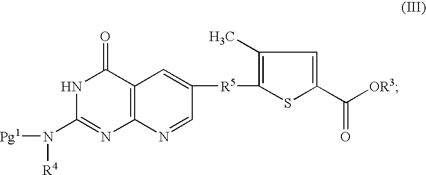
wherein R³ is a moiety that together with the attached CO₂ forms a readily hydrolyzable ester group;
R⁴ is H;
Pg¹ is an amino protecting group; or
Pg¹ can optionally be taken together with R⁴ and the nitrogen to which Pg¹ and R⁴ are attached to form (i) an imine; or (ii) a fused or bridged bicyclic ring or a spirocyclic ring, wherein said ring is saturated and contains from 5 to 12 carbon atoms in which up to 2 carbon atoms are optionally replaced with a hetero moiety selected from O, S(O)ⱼ wherein j is an integer from 0 to 2, and —NR⁸—, provided that two O atoms, two S(O)ⱼ moieties, or an O atom and a S(O)ⱼ moiety are not attached directly to each other;
R⁵ is selected from the group consisting of —C≡C— and —CH═CH—; and
R⁸ is independently H or C₁-C₆ alkyl;
with a hydrogenating agent in the presence of a transition-metal catalyst;
to form a compound of the formula (IV): 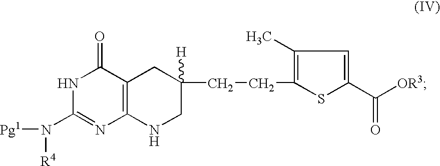
wherein each of said Pg¹, R³, and R⁴ are as described above;
(b) reacting said compound of formula (IV), with a base under an aqueous condition to obtain a compound of the formula (II): 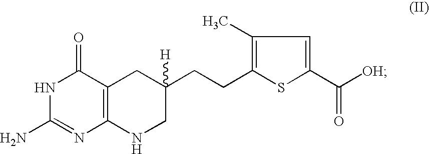
or a salt thereof; and
(c) reacting said compound of the formula (II) with a coupling agent and a base, followed by an L-glutamic acid diester salt, to form said compound of the formula (I).

2. A method according to claim 1, wherein said step (c) comprises the following steps:
(c-1) reacting said compound of the formula (II) with a coupling agent and a base, followed by an L-glutamic acid diester salt, to form a diastereomeric mixture of compounds of the formula (Ib): 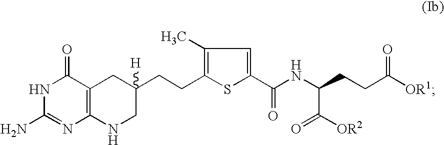
or salts thereof, wherein each of R¹ and R² are as described above;
(c-2) separating through a separation means said diastereomeric mixture of compounds of the formula (Ib) into compounds (Ic) and (Id): 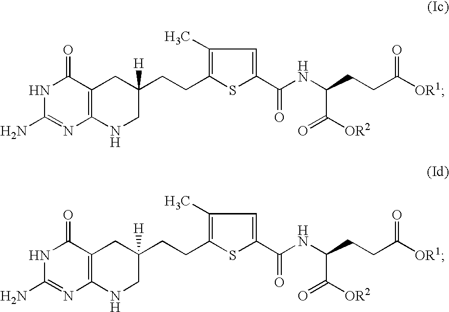
wherein each of R¹ and R² are as described above; and
(c-3) reacting said compound (Ic) with a suitable deprotecting agent to form an optically active compound or salt of the formula (Ia): 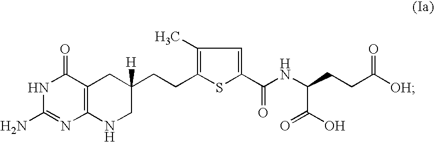
(c-4) reacting said compound (Id) with a suitable deprotecting agent to form an optically active compound or salt of the formula (Ie):

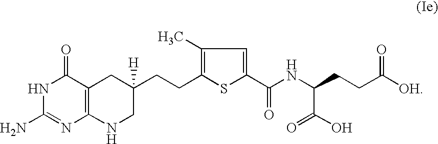

3. A method according to claim 1, wherein in the compound of the formula (III), said R⁵ is —C≡C—.

4. A method according to claim 1, wherein in step (b), said base is a hydroxide ion.

5. A method according to claim 1, wherein the method further comprises the following steps of preparing said compound of formula (III):
(d-1) reacting a compound of the formula (Va): 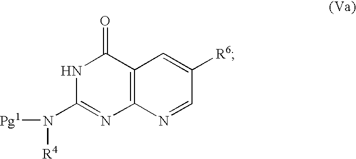
wherein R⁵ is halo, triflate or other activating group;
with a compound of formula (VIb), in the presence of a catalyst, a base, and a solvent, 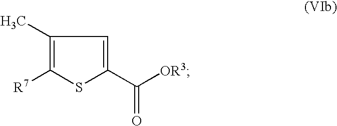
wherein R⁷ is —C≡CH; and R³ is a moiety that together with the attached CO₂ forms a readily hydrolyzable ester group.

6. A method according to claim 1, wherein the method further comprises the following steps of preparing said compound of formula (III):
(d-2) reacting a compound of the formula (Va): 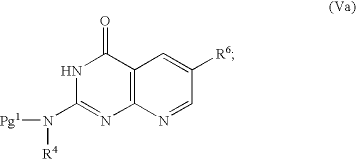
wherein R⁶ is halo, triflate or other activating group;
with a compound of formula (VIa), in the presence of a catalyst, a base, and a solvent: 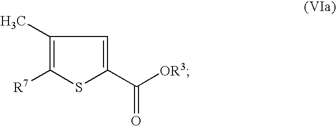
wherein R⁷ is —CH═CH₂; and R³ is a moiety that together with the attached CO₂ forms a readily hydrolyzable ester group.

7. A method according to claim 5, wherein said catalyst is palladium acetate.

8. A method according to claim 6, wherein said catalyst is palladium acetate.

9. A method according to claim 1, wherein the method further comprises the following steps of preparing said compound of formula (III):
(d-3) reacting a compound of the formula (Vb): 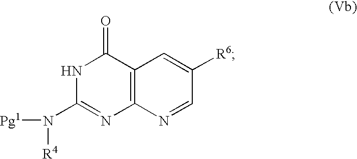
wherein R⁶ is —C≡CH, and Pg¹ and R⁴ are as described above;
with a compound of formula (VIc), in the presence of a catalyst, a base, and a solvent: 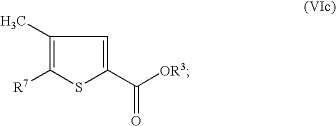
wherein R⁷ is halo, triflate or other activating group; and R³ is as described above.

10. A method according to claim 1, wherein the method further comprises the following steps of preparing said compound of formula (III):
(d-4) reacting a compound of the formula (Vc): 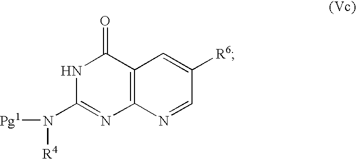
wherein R⁶ is —CH═CH₂, and Pg¹ and R⁴ are as described above;
with a compound of formula (VIc), in the presence of a catalyst, a base, and a solvent: 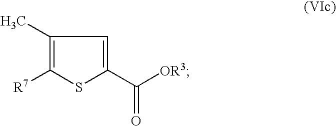
wherein R⁷ is halo, triflate or other activating group; and R³ is as described above.

11. A method according to claim 5, wherein said Pg¹ is (CH₃)₃—C—(C═O)—.

12. A method according to claim 6, wherein said Pg¹ is (CH₃)₃—C—(C═O)—.

13. A method according to claim 9, wherein said Pg¹ is (CH₃)₃—C—(C═O)—.

14. A method according to claim 10, wherein said Pg¹ is (CH₃)₃—C—(C═O)—.

15. A method according to claim 9, wherein the method further comprises the following steps of preparing said compound of formula (Vb):
(e-1) reacting said compound of the formula (Va): 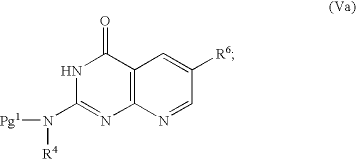
wherein R⁶ is halo, triflate or other activating group;
with a reagent having a formula H—C≡—C-Pg², to form a compound of the formula 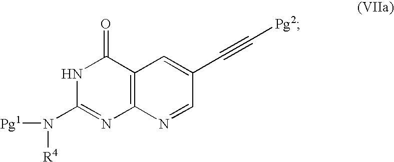
wherein Pg² is a protecting group; and
(f reacting said compound of formula (VIIa) with a deprotecting agent in a solvent to obtain said compound of the formula (Vb).

16. A method according to claim 10, wherein the method further comprises the following steps of preparing said compound of formula (Vc), as described above,
(e-2) reacting said compound of formula (Va):
reacting said compound of the formula (Va): 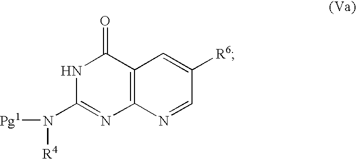
wherein R⁶ is halo, triflate or other activating group;
with a reagent having a formula CH₂═CH₂, in the presence of a base, catalyst and solvent, to form said compound of the formula (Vc).

17. A compound of formula (Ib) or a salt thereof: 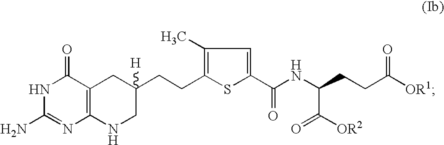
wherein each of R¹ and R² are independently a moiety that together with the attached CO₂ forms a readily hydrolyzable ester group.

18. A compound of formula (III) or a salt thereof: 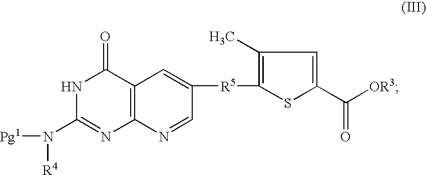
wherein R³ is a moiety that together with the attached CO₂ forms a readily hydrolyzable ester group;
Pg¹ is an amino protecting group;
R⁴ is H; or
Pg¹ can optionally be taken together with R⁴ and the nitrogen to which Pg¹ and R⁴ are attached to form (i) an imine; or (ii) a fused or bridged bicyclic ring or a spirocyclic ring, wherein said ring is saturated and contains from 5 to 12 carbon atoms in which up to 2 carbon atoms are optionally replaced with a hetero moiety selected from O, S(O)ⱼ wherein j is an integer from 0 to 2, and —NR⁸—, provided that two O atoms, two S(O)ⱼ moieties, or an O atom and a S(O)ⱼ moiety are not attached directly to each other;
R⁵ is selected from the group consisting of —C≡—C—and —CH═CH—; and
R⁸ is independently H or C₁-C₆ alkyl.

19. A compound of formula (IV), a salt thereof, an enantiomeric mixture thereof, or pure enantiomers thereof: 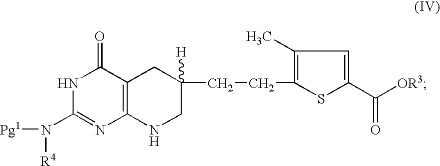
R³ is a moiety that together with the attached CO₂ forms a readily hydrolyzable ester group;
Pg¹ is amino protecting group;
R⁴ is H; or
Pg¹ can optionally be taken together with R⁴ and the nitrogen to which Pg¹ and R⁴ are attached to form (i) an imine; or (ii) a fused or bridged bicyclic ring or a spirocyclic ring, wherein said ring is saturated and contains from 5 to 12 carbon atoms in which up to 2 carbon atoms are optionally replaced with a hetero moiety selected from O, S(O)ⱼ wherein j is an integer from 0 to 2, and —NR⁸—, provided that two O atoms, two S(O)ⱼ moieties, or an O atom and a S(O)ⱼ moiety are not attached directly to each other; and R⁸ is independently H or C₁-C₆ alkyl.
